(19) United States
(12) Patent Application Publication
(10) Pub. No.: US 2024/0164861 A1
(43) Pub. Date: May 23, 2024

(54) DISPOSABLE STETHOSCOPE COVER AND ATTACHMENT MECHANISM

(71) Applicant: 3D HOLDING COMPANY, LLC, Scottsdale, AZ (US)

(72) Inventors: Calvin DUDLEY, Lynchburg, VA (US); Justin D. ANDERSON, Scottsdale, AZ (US)

(21) Appl. No.: 18/552,699

(22) PCT Filed: Apr. 28, 2022
(86) PCT No.: PCT/US2022/026718

§ 371 Date: Sep. 27, 2023

Related U.S. Application Data
(60) Provisional application No. 63/180,940, filed on Apr. 28, 2021.

(51) Int. Cl. A61B 50/00

(52) U.S. Cl. CPC A61B 50/00; A61B 2050/007

(57) ABSTRACT

A disposable stethoscope cover system includes a clamp which is configured to be mechanically attachable to stethoscope tubing; and a disposable cover which is configured to be mechanically attachable to the clamp. According to an embodiment, a clip and clamp mechanism is configured to releasably attach a stethoscope cover to a stethoscope and includes a clamp mechanism configured to be press fit to the stethoscope tubing to connect the clamp and clamp mechanism to the stethoscope; and a clip mechanism which is configured to releasably grasp a portion of the stethoscope cover.

### RELATED APPLICATION

This application is related to, and claims priority from, U.S. Provisional Patent Application No. 63/180,940, entitled Disposable Stethoscope Cover and Attachment Mechanism, filed on Apr. 28, 2021, the entire disclosure of which is hereby incorporated by reference.

### TECHNICAL FIELD

Embodiments described herein relate in general to stethoscopes and, more particularly, disposable stethoscope cover attachment mechanisms.

### BACKGROUND

It is estimated that 1.7 million patients develop healthcare-associated infections (HAIs) in the USA each year, resulting in approximately 100,000 deaths annually. The financial burden of HAIs to the US health care system is enormous, ranging from $28 billion to $45 billion per year. It is believed that a large proportion of HAls are preventable. Thus there has been increasing intensity and pressure on hospitals to reduce their HAI rate.one of the major efforts has been to educate care givers on hand sanitization and personal protective equipment (PPE) such as facemasks, gowns and gloves.

However relatively little has been said about the stethoscope which can be considered to be an extension of the care giver. Stethoscopes are widely recognized as harboring and transmitted the harmful bacteria and viruses that cause HAls. The diaphragm of the stethoscope and the rubber tubing of the stethoscope have both been shown to harbor bacterial levels comparable to a clinician's hands after a physical examination. The implication of this is that the stethoscope should be treated as an extension of the caregiver's hands in the context of preventing HAIs potentially caused by stethoscopes.

While there has been widespread education and standards on hand washing and PPE, stethoscope hygiene has been largely overlooked. Studies have shown that the amount of effort given to stethoscope sanitation is very low among most caregivers. For example, most surveys indicate that 70-90% of physicians do not systematically disinfect their stethoscopes between patient examinations and, if they do disinfect their stethoscopes between examinations using standard disinfecting wipes, this is not sufficient to adequately eliminate bacteria. The time required to adequately disinfect stethoscopes is also cited as a barrier, since the medical examination field is notoriously time/volume driven. There is currently no universal standardized stethoscope hygiene protocol.

In attempts to prevent stethoscopes from transmitting HAls, some health care systems utilize disposable stethoscopes. However disposable stethoscopes are relatively costly and generally have poor acoustics relative to non-disposable stethoscopes, which make them suboptimal for examining patients.

There has been some development of stethoscope covers that overcome the above-described limitations to stethoscope sanitation. However they have not been widely adopted in the medical field, possibly due to their design and functionality. For example, some stethoscope covers only cover the bell or diaphragm of the stethoscope which does not account for contamination of the rubber tubing. Examples of such stethoscope covers can be seen in U.S. Patent Application Publications No. 2017/0095224 and 2017/0020618. There are few options available for full length stethoscope covers and they are limited by cost, ease of use, packaging and dispensing, mechanism of attachment to the stethoscope and composition material.

Accordingly, it would be desirable to provide disposable stethoscope covers and attachment mechanisms which address these and other problems.

### SUMMARY

According to embodiments, disposable stethoscope covers and attachment mechanisms therefore which are easy to use and provide firm and complete hygienic covering of stethoscopes are described.

According to an embodiment, a disposable stethoscope cover system includes a clamp which is configured to be mechanically attachable to stethoscope tubing; and a disposable cover which is configured to be mechanically attachable to the clamp.

According to another embodiment, a stethoscope clamp is configured to be mechanically attachable to stethoscope tubing; and is configured to be mechanically attachable to a disposable cover.

According to yet another embodiment, a disposable stethoscope cover which has a first portion which is a substantially planar upper surface upon which at least one of an opening or Velcro® type hook and loop fastener material are disposed, and a second portion which is a sleeve with an opening into which a stethoscope diaphragm and tubing can be inserted.

According to a further embodiment, a clip and clamp mechanism which is configured to releasably attach a stethoscope cover to a stethoscope includes a clamp mechanism, configured to be press fit to the stethoscope tubing to connect the clamp and clamp mechanism to the stethoscope; and a clip mechanism which is configured to releasably grasp a portion of the stethoscope cover.

According to another embodiment, a stethoscope cover system includes a stethoscope including a bell and stethoscope tubing; a stethoscope cover; a clip and clamp mechanism which is configured to releasably attach the stethoscope cover to the stethoscope including: a clamp mechanism, configured to be press fit to the stethoscope tubing to connect the clamp and clamp mechanism to the stethoscope; and a clip mechanism which is configured to releasably grasp a portion of the stethoscope cover; wherein the stethoscope can be covered by the stethoscope cover by lowering the bell of the stethoscope into an opening in a top portion of the stethoscope cover.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 illustrates a stethoscope;

FIGS. 2A and 2B depict a disposable stethoscope cover system according to a first embodiment;

FIGS. 3A and 3B show a hook clamp according to the first embodiment;

FIG. 4 shows a disposable stethoscope cover system according to a second embodiment;

FIGS. 5A and 5B show a clamp for attaching a disposable cover to a stethoscope according to the second embodiment;

FIGS. 6A and 6B depict a disposable stethoscope cover system according to a third embodiment;

FIG. 7 shows a disposable stethoscope cover system according to an embodiment;

FIGS. 8-9 show a clamp for attaching a disposable cover to a stethoscope according to a fourth embodiment;

FIG. 10 shows a disposable stethoscope cover according to an embodiment;

FIG. 11 depicts a clamp for attaching a disposable cover to a stethoscope according to a fifth embodiment; and

FIG. 12 depicts a clamp for attaching a disposable cover to a stethoscope according to a sixth embodiment.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. The embodiments to be discussed next are not limited to the configurations described below, but may be extended to other arrangements as discussed later.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to various embodiments described herein, disposable stethoscope covers are provided in a simple and affordable design which enable the users to put on and take of the stethoscope cover easily and quickly. According to some embodiments, the stethoscope cover can have the same composition as a standard medical glove and can be packaged in a similar manner. The embodiments described herein eliminate the potential barriers to widespread use of stethoscope covers to reduce the morbidity, mortality and significant cost of HAIs.

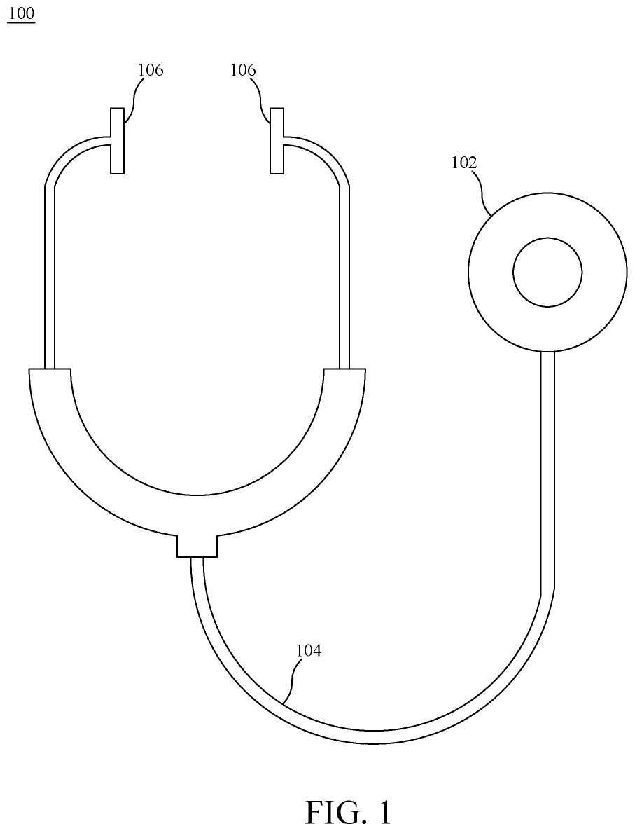

Prior to describing these embodiments in detail, it will be useful to first discuss the structure of an exemplary stethoscope to understand the terminology associated therewith. An exemplary stethoscope 100 is shown in FIG. 1. Acoustic stethoscopes operate by transmitting sound from the chest piece 102, via air-filled hollow tubes 104, to the listener's ears via earpieces 106. The chest piece 102 usually consists of two sides either of which can be placed against the patient for sensing sound: specifically, a diaphragm or bell. If the diaphragm is placed on the patient, body sounds vibrate the diaphragm, creating acoustic pressure waves which travel up the tubing to the listener's ears. If the bell is placed on the patient, the vibrations of the skin directly produce acoustic pressure waves traveling up to the listener's ears. The bell transmits low frequency sounds, while the diaphragm transmits higher frequency sounds. To deliver the acoustic energy primarily to either the bell or diaphragm, the tube connecting into the chamber between bell and diaphragm is open on only one side and can rotate. Embodiments described below seek to provide such stethoscopes with disposable covers which are firmly, mechanically attached to the stethoscope and yet easy to both attach and remove.

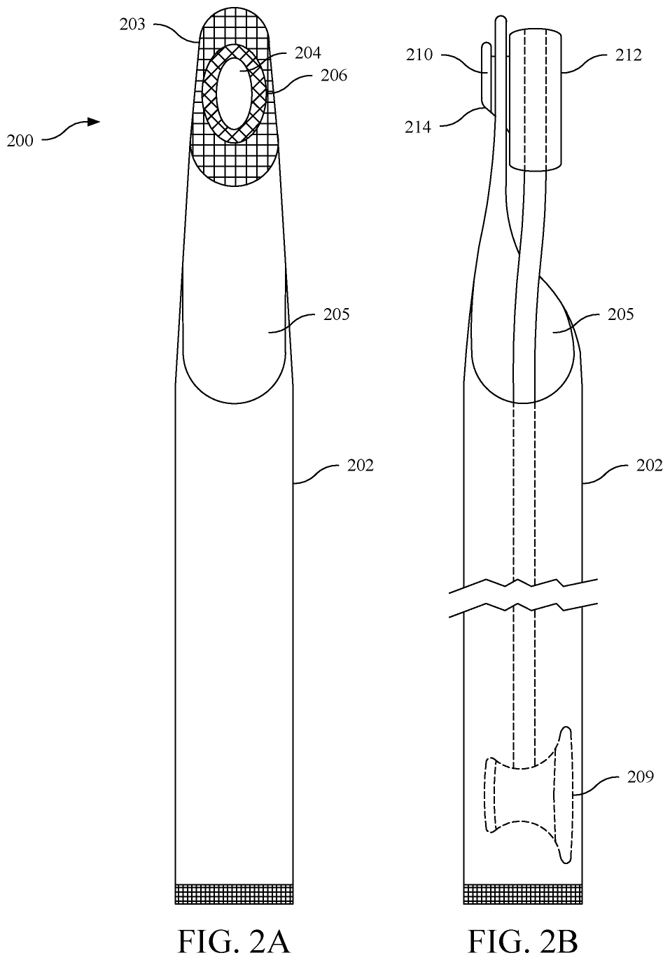

FIGS. 2A-2B depict a first embodiment of a disposable stethoscope cover and attachment mechanism. FIG. 2A depicts a front view of just the cover 200 without the stethoscope. In this embodiment the cover 200 can be fabricated from, for example, latex, nitrile, vinyl or cloth, among other materials. There are two primary sections to the cover 200: a bottom sleeve portion 202 into which the stethoscope and tubing can be inserted and covered, and a top portion 203 which includes a hook hanging hole or opening 204. Between the top portion 203 and the bottom sleeve portion 202, there is an opening 205 disposed where the top portion 203 transitions from being a substantially flat, planar surface into a tube. The stethoscope can be inserted into the opening 205 and dropped through the tube that forms the bottom sleeve portion 202 to cover the stethoscope. The top portion 203 can have a paper tab or reinforcement area 206 around the opening 204. The bottom sleeve portion should be dimensioned to be able to easily accept feeding of the bell and/or diaphragm portion of a stethoscope through the opening 205.

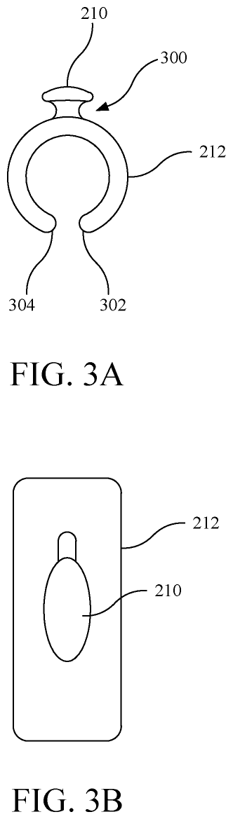

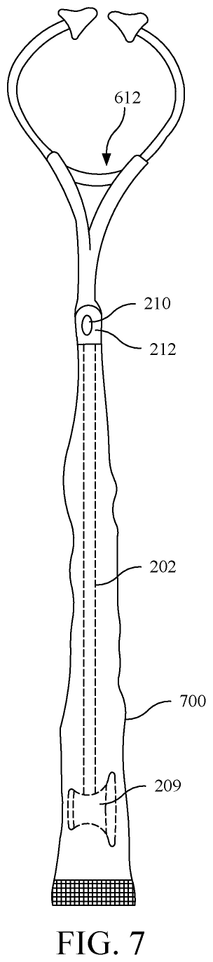

FIG. 2B shows a side view of the cover 200 attached to a stethoscope with the bell and/or diaphragm 209 of the stethoscope, and most of the tubing, inside of the bottom sleeve portion 202. The opening 204 is looped over a hook 210 to physically connect the cover 200 to the stethoscope. As will be more fully described below, the hook 210 is connected to a hook clamp 212 which clamps onto the stethoscope tubing at a location which is proximate the juncture where the two earpiece wires diverge from the tubing which is connected to the bell and/or diaphragm 209 of the stethoscope. See also FIG. 7. The hook 210 and the hook clamp 212 can be integrally formed together or fabricated as separate parts and create a groove between the hook 210 and hook clamp to retain the cover 200. The hook clamp 212 (shown in more detail in FIGS. 3A and 3B) can be fabricated from plastic and has an inner diameter that is sized to compress onto (or be popped onto) the tube of the stethoscope. The bottom edge of the hook 210 can have an angled surface, e.g., about 45 degrees relative to the cover 200 when installed on the hook 210, to facilitate easy removal of the cover 200 from the hook clamp 212.

FIGS. 3A and 3B depict a top view and a front view, respectively, of the hook 210/hook clamp 212 combination. In FIG. 3A, the retaining groove 300 formed between hook 210 and hook clamp 212 can be seen more clearly. The tips 302, 304 at the ends of the ring-shaped hook clamp 212 can be smoothed so as to not damage the stethoscope tubing when the hook clamp 212 is pressed onto (or removed from) the tubing.

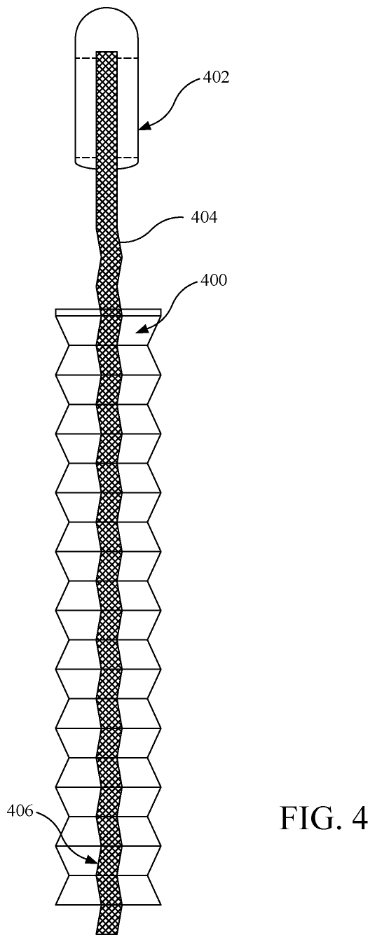

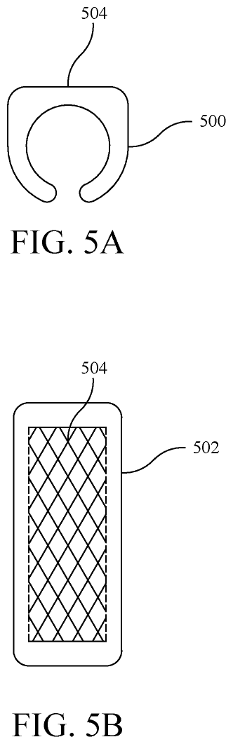

The foregoing embodiment of FIGS. 2A-3B used a hook attached to the stethoscope and a corresponding opening in the cover to mechanically fasten the cover 200 to the stethoscope. However, according to a second embodiment, Velcro® type hook and loop fastener can be used instead. An example of this second embodiment is depicted in FIG. 4. Therein, the stethoscope cover 400 can be made from latex, nitrile or cloth and, in this embodiment, is shown having fold lines that enable the cover 400 to be folded up like an accordion. A connector tab 402 can have the Velcro® type hook and loop fastener material on the other side of the surface shown in FIG. 4 (see FIG. 5B) and can be connected to the cover 400 by a cloth connector string 404 which can also include a tab 406 at the bottom thereof which can be used to pull open the cover 400 for insertion of the stethoscope into an opening at the top portion of the cover 400.

FIG. 5A shows the holding clamp 500 which operates in conjunction with the connector tab 502 (FIG. 5B) to mechanically attach the combination of the holding clamp 500, the connector tab 402, 502 and cover 400 to a stethoscope in a manner similar to that described above with respect to the first embodiment. The Velcro® type hook and loop fastener 504 is positioned on both an outer surface of the holding clamp 500 and a surface of the connector tab 502 such that when these two surfaces are pressed together, the cover 400 becomes removably attached to the clamp 500 which can be press fit over the tube of a stethoscope.

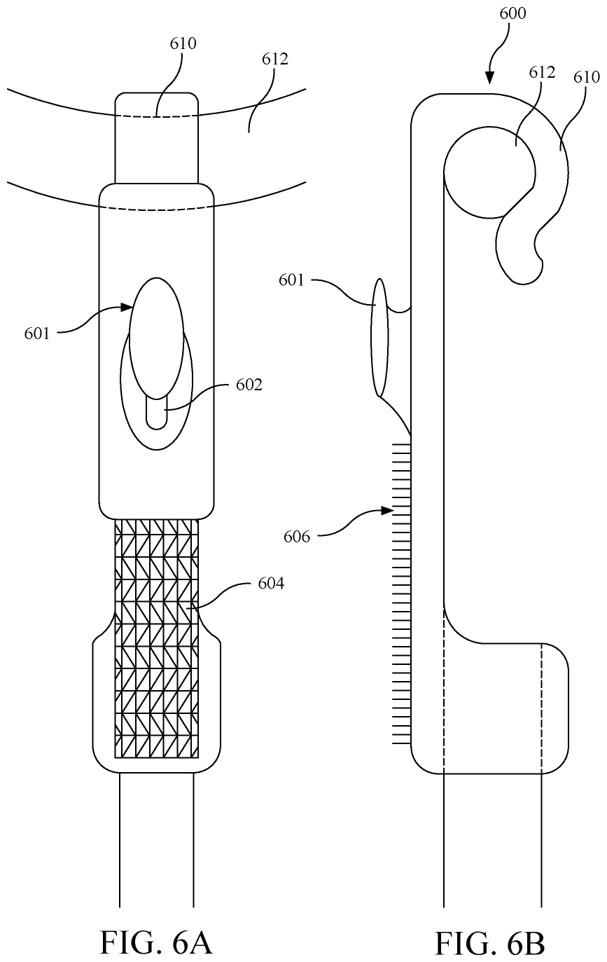

According to a third embodiment, both the hang hook and opening of the first embodiment and the Velcro® type hook and loop fastener of the second embodiment can be used to removably attach the cover to the clamp (and hence the stethoscope). An example of this third embodiment is shown in FIGS. 6A and 6B. FIG. 6A is a front view showing the clamp connected to the cover and to the stethoscope, while FIG. 6B is a side view of just the clamp component. Therein, in a manner similar to that of the first embodiment, the clamp 600 includes a hook element 601 onto which the cover hangs by looping opening 602 onto the hook 601. Additionally, both the cover and the clamp 600 include portions 604 and 606, respectively, which include Velcro® type hook and loop fastener material which when mated and pressed together provide a second mechanical fastening of the cover to the clamp (and hence the stethoscope) in addition to the hang hook and opening.

In addition, this embodiment includes an optional, third mechanical connection in the form of a second hook 610 which loops over top of a horizontal piece of tubing 612 on the stethoscope. While not all stethoscopes include this horizontal piece of tubing 612 an example is shown in FIG. 7, although in this illustration a hook clamp 212 and hook 210 are shown as clamping onto the stethoscope tubing 202 to enable attachment of a disposable stethoscope cover 700.

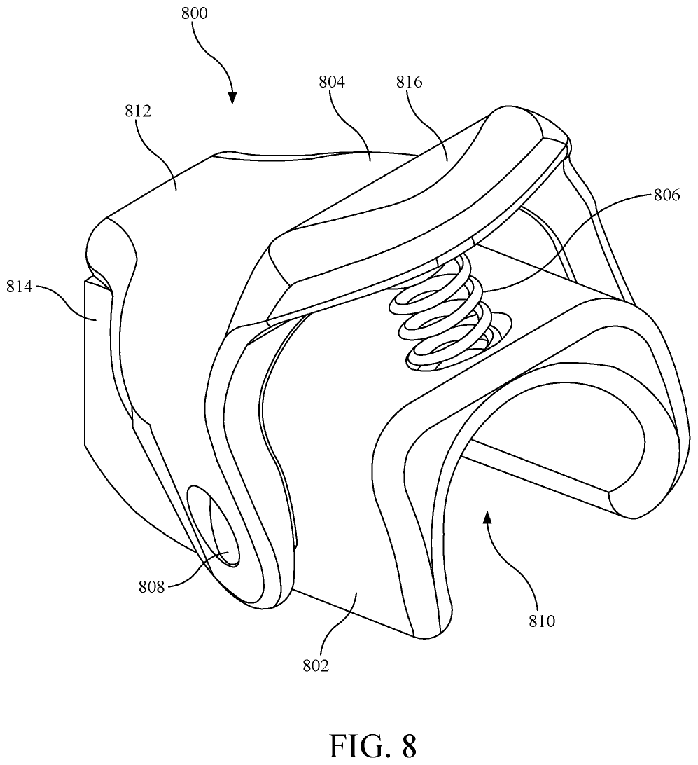

FIG. 8 shows an isometric rear view of clamp and clamp mechanism 800 for attaching a disposable stethoscope cover to a stethoscope according to a fourth embodiment. The clamp and clamp mechanism 800 includes three elements in this embodiment: a stethoscope clamp portion 802, a rocker clamp portion 804, and a spring 806 disposed between the clamp portion 802 and the rocker clamp portion 804. The rocker clamp portion 804 is rotatably mounted on two pivot pins 808 (only one of which is visible in FIG. 8, the other pivot pin 808 is disposed on the non-visible side of the clamp portion 802 directly across from the visible pivot pin 808). The clamp portion 802 has a semi-cylindrical opening 810 formed therein which is sized to be just slightly smaller than the diameter of the stethoscope tubing 202 to enable the clamp portion of be press fit onto the stethoscope tubing 202. Since stethoscope tubing can come in various sizes, i.e., diameters, according to different embodiments the opening 810 can have different sizes to render an appropriate press fit with the differently sized stethoscope tubing.

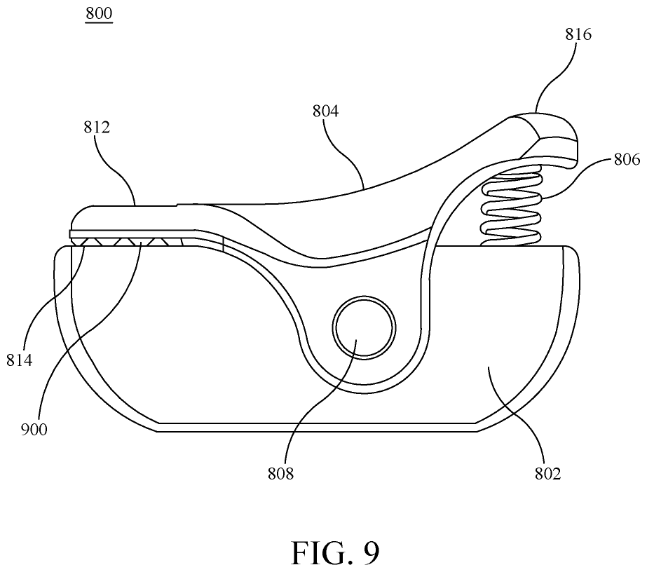

The rocker clamp 804 is operable to releasably clamp a disposable stethoscope cover, e.g., stethoscope cover 1000 shown in FIG. 10, to the clamp and clamp mechanism 800. As also seen in the side view of FIG. 9, the spring 806 biases the rocker clamp arm 804 into its clamped position where a front surface 812 of the rocker clamp arm 804 is pressed against a top surface 814 of the clamp portion 802. As also seen in the side view of FIG. 9, to attach a disposable stethoscope cover 1000 to the clamp and clamp mechanism 800, a user presses down on the portion 816 of the rocker clamp 804 proximate the spring 806 with enough pressure to pivot the rocker clamp 804 into a position where the front surface 812 of the rocker clamp arm is raised away from the top surface 814 of the clamp portion 802. This enables the user to insert the opening between surfaces 812 and 814 of the clamp and clamp mechanism 800 through an opening 1002 in the disposable stethoscope cover 1000 and to then grab/clamp a wall 1004 of the disposable stethoscope cover 1000 by releasing pressure on the rocker arm portion 816 such that the rocker clamp 804 returns to its biased, closed position and clamps the cover 1000 between surfaces 812 and 814. Teeth 900 can be provided to the surfaces 812 and/or 814 in order to more tightly grab the disposable stethoscope cover.

The stethoscope cover 1000 may be made of nitrile or any other suitable material and formed as a hollow tube with a rolled lip 1106 and being flared outwardly at 1104 to enable the bell 209 of the stethoscope to be more easily inserted into the opening 1002. Alternatively, the disposable stethoscope cover can be fabricated to have an oval-shaped cross-section and sized to enable a dual head or double bell stethoscope to be covered. Although shown as a spring 806, any other type of biasing mechanism can be used to bias the rocker clamp into its closed position.

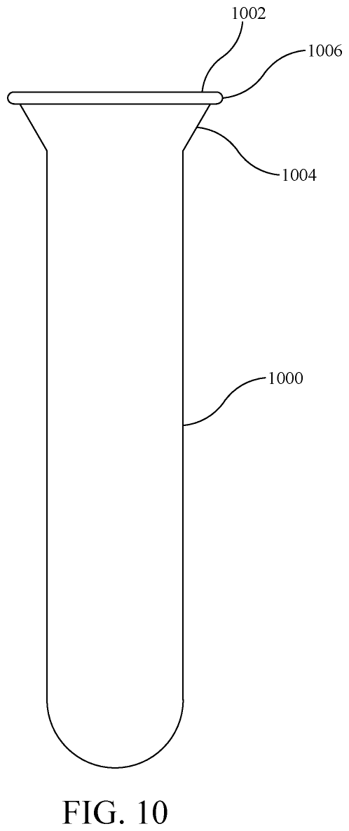

FIG. 11 shows another embodiment of a mechanism 1100 for attaching a stethoscope cover to stethoscope. Unlike the embodiment of FIG. 8 and FIG. 9, wherein a surface 814 of the stethoscope clamp 802 cooperates with a surface 812 of a rocker clamp 804 to grip the stethoscope cover when the mechanism 800 is biased into its closed position, the embodiment of FIG. 11 includes a two-part clamp having a pivoting arm 1102 and a non-pivoting arm 1104, e.g., made out of stainless steel, attached to a stethoscope clamp 1106, which can be made out of plastic. The two-part clamp 1102, 1104 and the stethoscope clamp 1106 can be connected to one another using a rivet 1108, pin or the like. Like the clamp 802, 810 in the embodiment of FIG. 8, the stethoscope clamp 1106 can be fabricated as a U-shaped plastic piece which is sized to be pressure fit onto stethoscope tubing. In operation, upper clamp part 1102 can be depressed at end 1110 to raise the other end of the clamp 1112 so that a side wall of the disposable stethoscope cover can be inserted between end 1112 and portion 1114 of the clamp. Depression of end 1110 overcomes a biasing force which otherwise keeps the mechanism 1100 in its closed position. In this embodiment, the biasing force can be provided by a coil spring (not shown) which is wrapped around pivot pin 1116. Note that in this embodiment, the center of rotation of the pivot pin 1116 can be placed proximate an edge of the U-shaped stethoscope clamp 1106 and the end 1110 can be disposed over a center portion of the stethoscope clamp 1106. In this way, when a user is pressing his or her left index finger on the end 1110 to open the clip 1102, his or her left thumb can press the stethoscope tubing into the U-shaped clamp 1116 to secure the stethoscope to the clip and clamp mechanism, while at the same time pull the disposable cover over the stethoscope bell and insert one end of the disposable cover into the opening created between elements 1112 and 1114. The two pressure points created by the left index finger on the top of the clip and clamp mechanism and by the left thumb on the bottom of the clip and clamp mechanism to press fit the tubing into the clamp are thus aligned with one another. The user can then remove the pressure from the left index finger to clip the disposable stethoscope cover to the clip and clamp mechanism with it covering the stethoscope and assembly is complete.

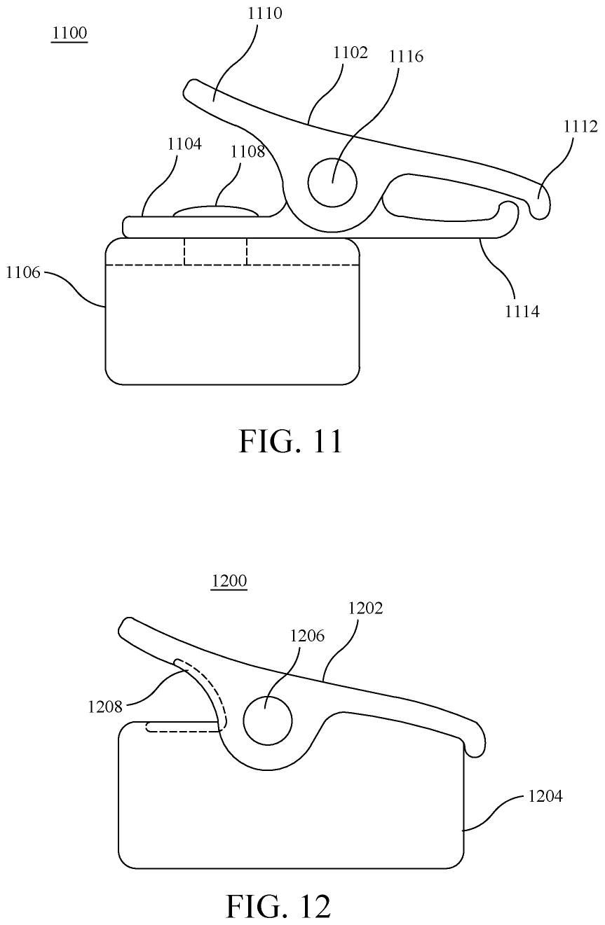

FIG. 12 illustrates a clamp 1200 for attaching a disposable cover to a stethoscope according to a sixth embodiment. Like the embodiment of FIG. 8 and FIG. 9, this clamp 1200 includes a rocker clamp arm 1202 which is rotatably mounted via pivot pin 1206 to a U-shaped clamp 1204 that is configured to be press fit onto stethoscope tubing. Unlike the embodiment of FIG. 8 and FIG. 9, the rocker clamp arm 1202 is biased into the closed position by a flat spring (not shown) which his shaped to fit into the pocket 1208 which is disposed within a portion of the rocker clamp arm 1202 and the U-shaped clamp 1206.

According to embodiments, a disposable stethoscope cover system includes a clip which is configured to be mechanically attachable to stethoscope tubing; and a disposable cover which is configured to be mechanically attachable to the clip. According to one embodiment, the clip is configured to be press fit onto the stethoscope tubing and includes a hook and wherein the disposable cover include an opening which is configured to be slid over the hook. According to another embodiment, the clip is configured to be press fit onto the stethoscope tubing wherein both the clip and the disposable cover have surfaces which include Velcro® type hook and loop fastener material which can be pressed together to mechanically attach the cover to the clip. According to another embodiment, the clip is configured to be press fit onto the stethoscope tubing and includes a hook and wherein the disposable cover include an opening which is configured to be slid over the hook and further wherein both the clip and the disposable cover have surfaces which include Velcro® type hook and loop fastener material which can be pressed together to mechanically attach the cover to the clip. The disposable cover has a first portion which is a substantially planar upper surface upon which at least one of an opening or Velcro® type hook and loop fastener material are disposed, and a second portion which is a sleeve with an opening into which a stethoscope diaphragm and tubing can be inserted. The disposable cover is fabricated from either latex, nitrile, vinyl or cloth.

It should be understood that this description is not intended to limit the invention. On the contrary, the embodiments are intended to cover alternatives, modifications and equivalents, which are included in the spirit and scope of the invention. Further, in the detailed description of the embodiments, numerous specific details are set forth in order to provide a comprehensive understanding of the invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A clip and clamp mechanism which is configured to releasably attach a stethoscope cover to a stethoscope comprising:
a clamp mechanism, configured to be press fit to the stethoscope tubing to connect the clamp and clamp mechanism to the stethoscope; and
a clip mechanism which is configured to releasably grasp a portion of the stethoscope cover.

2. The clip and clamp mechanism of claim 1, wherein the clamp mechanism includes a rocker arm which is pivotally attached to the clamp mechanism and is pivotally biased into a closed position by a coil spring disposed between one end of the rocker arm and a top end surface of the clamp mechanism.

3. The clip and clamp mechanism of claim 1, wherein the clamp mechanism includes a rocker arm which is pivotally attached to the clamp mechanism and is pivotally biased into a closed position by a flat spring disposed in a pocket formed in the rocker arm and a top end surface of the clamp mechanism.

4. The clip and clamp mechanism of claim 2, wherein the clamp mechanism further includes another end of the rocker arm which is configured to be in contact with a top front surface of the clamp mechanism, wherein the stethoscope cover can be grasped between the another end of the rocker arm and the top front surface of the clamp mechanism.

5. The clip and clamp mechanism of claim 2, wherein the rocker arm is pivotally attached to the clamp mechanism by a combination of two holes in the rocker arm and two pins disposed on an outer surface of the clamp mechanism.

6. The clip and clamp mechanism of claim 1, wherein the clamp mechanism includes a pivoting arm and a non-pivoting arm which are pivotally attached together via a pivot pin and wherein the non-pivoting arm is fixedly attached to the clamp mechanism.

7. The clip and clamp mechanism of claim 2, wherein one end of the rocker arm is positioned above a central portion of the clamp mechanism.

8. The clip and clamp mechanism of claim 6, wherein one end of the pivoting arm is positioned above a central portion of the clamp mechanism.

9. A stethoscope cover system comprising:
a stethoscope including a bell and stethoscope tubing;
a stethoscope cover;
a clip and clamp mechanism which is configured to releasably attach the stethoscope cover to the stethoscope including:
a clamp mechanism, configured to be press fit to the stethoscope tubing to connect the clamp and clamp mechanism to the stethoscope; and
a clip mechanism which is configured to releasably grasp a portion of the stethoscope cover;
wherein the stethoscope can be covered by the stethoscope cover by lowering the bell of the stethoscope into an opening in a top portion of the stethoscope cover.

10. The stethoscope cover system of claim 9, wherein the clamp mechanism includes a rocker arm which is pivotally attached to the clamp mechanism and is pivotally biased into a closed position by a coil spring disposed between one end of the rocker arm and a top end surface of the clamp mechanism.

11. The stethoscope cover system of claim 9, wherein the clamp mechanism includes a rocker arm which is pivotally attached to the clamp mechanism and is pivotally biased into a closed position by a flat spring disposed in a pocket formed in the rocker arm and a top end surface of the clamp mechanism.

12. The stethoscope cover system of claim 10, wherein the clamp mechanism further includes another end of the rocker arm which is configured to be in contact with a top front surface of the clamp mechanism, wherein the stethoscope cover can be grasped between the another end of the rocker arm and the top front surface of the clamp mechanism.

13. The stethoscope over system of claim 10, wherein the rocker arm is pivotally attached to the clamp mechanism by a combination of two holes in the rocker arm and two pins disposed on an outer surface of the clamp mechanism.

14. The stethoscope cover system of claim 9, wherein the clamp mechanism includes a pivoting arm and a non-pivoting arm which are pivotally attached together via a pivot pin and wherein the non-pivoting arm is fixedly attached to the clamp mechanism.

15. The stethoscope cover system of claim 10, wherein one end of the rocker arm is positioned above a central portion of the clamp mechanism.

16. The stethoscope cover system of claim 14, wherein one end of the pivoting arm is positioned above a central portion of the clamp mechanism.

17. The disposable stethoscope cover system of claim 9, wherein the disposable cover is fabricated from either latex, nitrile, vinyl or cloth.
